(19) United States
(12) Patent Application Publication
(10) Pub. No.: US 2018/0303483 A1
(43) Pub. Date: Oct. 25, 2018

(54) DEVICE FOR ASSISTING HEALING OF SKIN WOUND

(71) Applicant: SICHUAN RUI ZE JIAN TECHNOLOGY CO. LTD., Chengdu, Sichuan (CN)

(72) Inventor: Xinping Zhang, Nanchong (CN)

(73) Assignee: Xiaoping Du, San Jose, CA (US)

(21) Appl. No.: 15/769,328

(22) PCT Filed: Oct. 18, 2016
(86) PCT No.: PCT/CN2016/102422

§ 371 Date: Apr. 18, 2018

(30) Foreign Application Priority Data
Oct. 19, 2015 (CN) 201510679792.4

(51) Int. Cl. A61B 17/08; A61M 1/00

(52) U.S. Cl. CPC A61B 17/08; A61M 1/0088; A61M 1/0023

(57) ABSTRACT

A device for assisting the healing of a skin wound applicable in the field of medical treatment and health care, and capable of timely cleansing a skin wound, eliminating a dead space in the skin wound, preventing infection and inflammation, facilitating healing of the skin wound, and preventing eversion and warp of edges of the skin wound during the healing process so that the healed skin is smooth and even. The device comprises: a sealing membrane (1); an evacuation tube (2); base straps (3); a sealing end (4); a flexible top cover (5); and a locking structure disposed on a negative-pressure chamber. The evacuation tube (2) is configured to draw air out of the negative-pressure chamber. A closed chamber can effectively insulate a skin wound (11) from the external environment and enable the skin wound (11) to remain clean. A negative-pressure environment is formed in the closed chamber, thereby preventing eversion and warp of edges of the skin wound during the healing process, and accordingly ensuring smoothness and evenness of skin during the healing process.

The present application is the U.S. national phase of PCT Application PCT/CN2016/102422 filed on Oct. 18, 2016, which claims a priority of the Chinese patent application No. 201510679792.4 filed on Oct. 19, 2015 and entitled “DEVICE FOR ASSISTING HEALING OF SKIN WOUND”, which is incorporated herein by reference in its entirety.

### TECHNICAL FIELD

This invention relates to an auxiliary healing device for skin wounds, especially an auxiliary healing device for skin wounds in the medical treatment field.

### BACKGROUND

The modern closure plan for surgical incisions or wounds must simultaneously consider relevant factors affecting wound healing such as: timely cleaning and drainage of exudate and sphacelus of wounds, elimination of dead spaces of skin incisions, flattening and alignment of skin edges of wounds, tension reduction of wounds, prevention of new wounds caused by suturing, and protection for moist coverage of wounds.

In prior art, after the wound is covered with negative-pressure dressing, it is only possible to clean and drain exudate and sphacelus, while requirements for alignment and flattening of skin edges of incisions and tension reduction of wounds (i.e. tension reduction at two sides of the skin wound) still remain unsatisfied. With the closing device for skin incisions in prior art, it is only possible to simply pull the two sides of the skin incision towards the centerline. In the pulling-together process, the device needs to rely on external forces and complicated pulling-together structures and steps. Moreover, stress on the skin is uneven; particularly, the irregular wounds with curved shapes will lead to some parts of the wounds excessively pulled while other parts unaligned. Besides, the device cannot clean and drain the exudate and sphacelus of skin incisions, nor eliminate dead spaces of skin incisions, nor satisfy requirements for suturing of irregular wounds with curved shapes, and nor protect wounds with moist coverage. No device in prior art considers about providing a wound support structure to ensure alignment and flattening of skin edges of wounds. Therefore, in prior art, there is no complete closure plan for skin wounds, i.e., a negative-pressure tension-relief suturing device that can timely clean and drain the exudate and sphacelus of wounds, eliminate dead spaces of incisions, ensure close alignment and flattening of skin edges of wounds, reduce tension evenly and effectively, avoid new wounds caused by suturing, and protect wounds with moist coverage.

### SUMMARY

The technical problems to be solved by the present invention are to provide an auxiliary healing device for skin wounds which can conduct negative-pressure cleaning and drainage of sphacelus of wounds, eliminate dead spaces of skin incisions, pull skin edges of wounds to align automatically, assist for alignment and flattening of skin edges of wounds, adapt to suturing for irregular wounds with curved shapes, and ensure moist coverage of wounds. Meanwhile, it also provides comfortable use and ease of maintenance.

The auxiliary healing device for skin wounds in the present invention for coping with the above technical problems comprises a sealing membrane, a drainage tube, base straps, sealing ends and an elastic upper cover; the elastic upper cover is connected above the base straps and the base straps are able to make linear movements relative to the elastic upper cover; the sealing ends are arranged at two ends of the elastic upper cover and the sealing ends, the base straps and the elastic upper cover form into a sealed cavity; the sealing membrane covers the sealing ends 4, the elastic upper cover and the base straps; and the drainage tube is communicated with the inside and outside of the sealed cavity.

Furthermore, sliding blocks are arranged on the elastic upper cover, guiding channels are arranged on the base straps, and the sliding blocks are embedded into the guiding channels.

Furthermore, rollers are arranged on the elastic upper cover, guiding rails are arranged on the base straps, and the rollers match with the guiding rails.

Furthermore, ratchets are arranged on the base straps, and stoppers are arranged on the elastic upper cover.

Furthermore, the device also comprises dressings and the dressings are arranged under the upper cover.

Furthermore, adhesive layers are arranged at lower parts of the base straps.

Furthermore, the elastic upper cover is of a segmental structure and the sealing membrane is of a segmental structure too.

Furthermore, the device also comprises a closing device for skin incision; the closing device for skin incision is connected to the base straps at two sides of the skin wound.

The beneficial effects of the present invention are: the sealed cavity can effectively insulate the skin wound from the outside environment and keep the skin wound clean. In this way, a negative-pressure environment is created inside the sealed cavity. In the negative-pressure environment, the base straps automatically move towards the middle of the wound and drive the skin at two sides of the wound to move towards the middle too, which pull the skin together. The elastic upper cover deforms to press closely against the base straps downward to form a datum plane; meanwhile the skin at two sides of the wound is pressed closely against the datum plane of the elastic upper covers. With the limitation function of the upper cover, the skin will not have eversion and warp, and will always remain smooth and regular during growth and healing

### BRIEF DESCRIPTION OF THE DRAWINGS

FIG. 1 is a front view of the device in the present invention, according to an embodiment.

FIG. 2 is a top view of the device in the present invention, according to an embodiment.

FIG. 3 is a diagram of the connection between the base straps and the elastic upper cover of the present invention, according to an embodiment.

Main components and locations with corresponding numbers in the drawings are as follows: sealing membrane 1, drainage tube 2, base straps 3, sealing ends 4, elastic upper cover 5, sliding blocks 6, guiding channels 7, ratchets 8, stoppers 9, adhesive layers 10 and wound 11.

### DETAILED DESCRIPTION OF THE PREFERRED EMBODIMENTS

The present invention is further described in combination with drawings as follows:

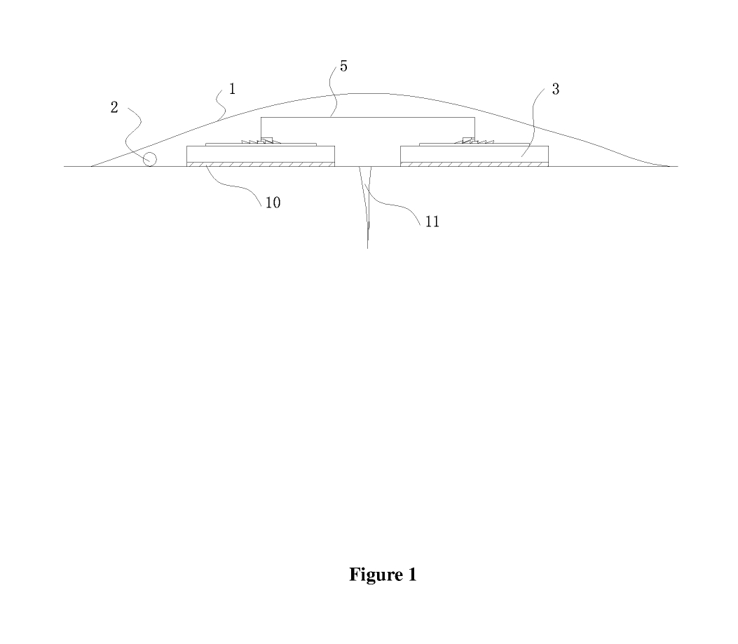

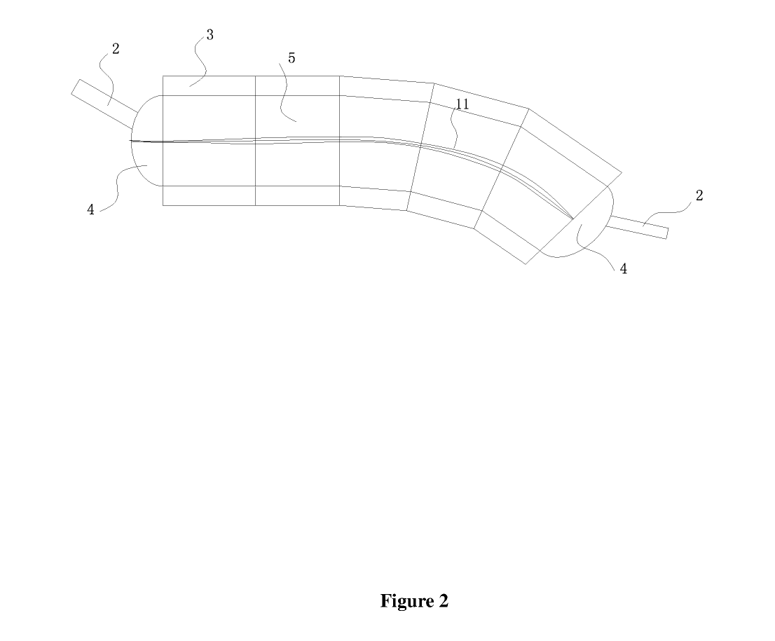

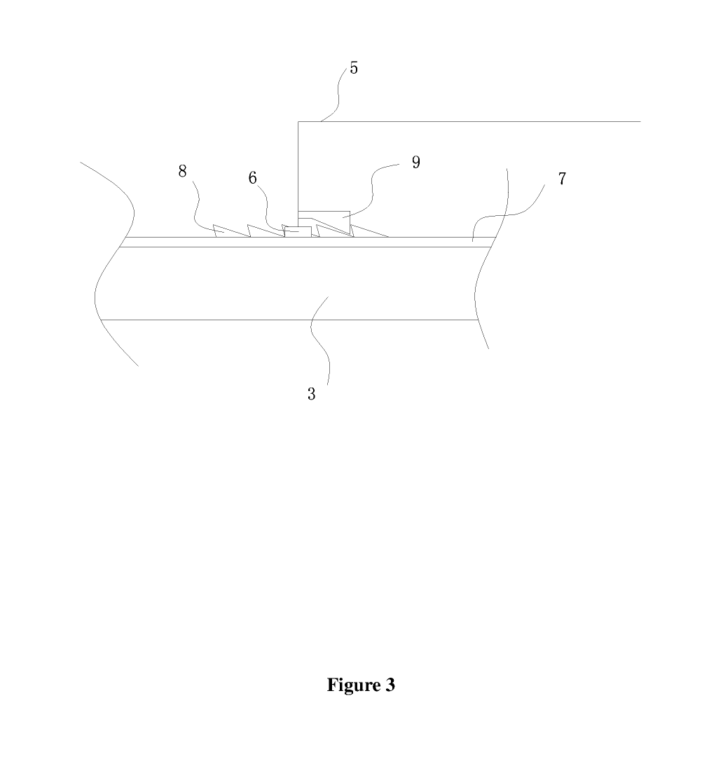

As shown in FIGS. 1, 2 and 3, the auxiliary healing device for skin wounds in the present invention comprises a sealing membrane 1, a drainage tube 2, base straps 3, sealing ends 4 and an elastic upper cover 5.

According to embodiment of the present invention, the elastic upper cover is connected above the base straps and the base straps are able to make linear movements relative to the elastic upper cover the sealing ends are arranged at two ends of the elastic upper cover and the sealing ends, the base straps and the elastic upper cover form into a sealed cavity; the sealing membrane covers the sealing ends; the elastic upper cover and the base straps; and the drainage tube is communicated with the inside and outside of the sealed cavity. The sealing ends, base straps 3 and elastic upper cover form into a sealed cavity.

A sealing membrane 1 is covered on the exterior to enhance sealing effect. The elastic upper cover 5 is made of an elastic material. When the device is applied, the base straps 3 is firstly bonded to the skin at two sides of the wound 11, and the air inside the sealed cavity is drawn out to the outside through the drainage tube 2 to create a negative-pressure environment inside the sealed cavity. The sealed cavity can effectively insulate the skin wound from the outside environment and keep the skin wound clean. Furthermore, when air is drawn out of the sealed cavity, the sealed cavity becomes a negative-pressure cavity. Under negative pressure, the base straps automatically move towards the middle of the wound and drive the skin at two sides of the wound to move towards the middle too, which pull the skin together and facilitate skin healing. Because the wound is in a negative-pressure environment, while pulling the skin together, the auxiliary healing device for the skin wound can help prevent dead spaces deep in the skin wound created during the pulling-together process.

In the pulling-together process, the drainage tube 2 is utilized to timely discharge exudate from the wound 11 to keep the wound clean, prevent skin infection and facilitate skin healing. Because the base straps 3 can make linear movements relative to the upper cover, the base straps are not affected by the upper cover when driving the skin wound move towards the middle under negative pressure, which will neither result in deformation of base straps during the movements nor wrinkle at two sides of the wound, thus increasing the healing efficacy of wound. Under negative pressure, the elastic upper cover 5 deforms to press closely against the base straps downward to form a datum plane, meanwhile the skin at two sides of the wound is pressed closely against the datum plane of the elastic upper covers. With the limitation function of the upper cover, the skin will not have eversion and warp, and will always remain smooth and regular during growth and healing, thus significantly improving healing efficacy of wound.

Sliding blocks 6 are arranged on the elastic upper cover 5, guiding channels 7 are arranged on the base straps 3, and the sliding blocks 6 are embedded into the guiding channels 7. Reliable connection and relative linear movements of the base straps 3 and the elastic upper cover 5 can be realized by the sliding blocks 6 and the guiding channels 7.

Rollers are arranged on the elastic upper cover 5, guiding rails are arranged on the base straps 3, and the rollers match with the guiding rails. The rollers and the guiding rails can be used as an alternative to realize the reliable connection and relative linear movement of the base straps 3 and the elastic upper cover 5.

Ratchets 8 are arranged on the base straps 3, and stoppers 9 are arranged on the elastic upper cover 5. External interference is inevitable when the base straps 3 pull the wound 11 together under negative pressure. At this time, the base straps 3 are prone to move backward and pull apart the wound which has partially healed. However, under cooperation of the ratchets 8 arranged on the base straps 3 and the stoppers 9 arranged on the elastic upper cover 5, it is possible to make the base straps move only towards the middle of the wound, not in the opposite direction, thus effectively preventing the base straps 3 from pulling apart the wound which has partially healed.

The device also comprises dressings and the dressings are arranged under the upper cover. The dressings are full of drugs which facilitate the healing of the wound 11, thus contributing to the rapid healing of the wound 11.

Adhesive layers 10 are arranged at lower parts of the base straps 3. In service, adhesive is applied to the lower parts of the base straps proximal to the skin, so as to pull the base straps and the skin at two sides of the wound together.

The elastic upper cover 5 is of a segmental structure and the sealing membrane 1 is of a segmental structure too. The elastic upper cover and the sealing membrane 1 are both of the segmental structure. The whole device is made up by assembling the multiple segments along the outline of the wound 11. By adopting the above mentioned structure, the device can adapt to the wound with irregular shape.

The device also comprises a closing device for skin incision; the closing device for skin incision is connected to the base straps 3 at two sides of the skin incision. The closing device for skin incision can pull the skin at two sides of the wound together.

What is claimed is:

1. An auxiliary healing device for skin wounds, comprising a sealing membrane , a drainage tube, base straps, sealing ends, and an elastic upper cover connected above the base straps, wherein
the base straps are able to make linear movements relative to the elastic upper cover;
the sealing ends are arranged at two ends of the elastic upper cover and the sealing ends, the base straps and the elastic upper cover form into a sealed cavity;
the sealing membrane covers the sealing ends, the elastic upper cover and the base straps; and
the drainage tube is communicated with the inside and outside of the sealed cavity.

2. The auxiliary healing device for skin wounds according to claim 1, wherein
one or more sliding blocks are arranged on the elastic upper cover;
one or more guiding channels are arranged on the base straps; and
the sliding blocks are embedded into the guiding channels.

3. The auxiliary healing device for skin wounds according to claim 1, wherein rollers are arranged on the elastic upper cover, guiding rails are arranged on the base straps, and the rollers match with the guiding rails.

4. The auxiliary healing device for skin wounds according to claim 1, wherein one or more ratchets are arranged on the base straps , and one or more stoppers are arranged on the elastic upper cover.

5. The auxiliary healing device for skin wounds according to claim 1, further comprising one or more dressings arranged under the upper cover.

6. The auxiliary healing device for skin wounds according to claim 1, characterized in that: adhesive layers are arranged at lower parts of the base straps (3).

7. The auxiliary healing device for skin wounds according to claim 1, characterized in that: the elastic upper cover (5) is of a segmental structure and the sealing membrane (1) is of a segmental structure too.

8. The negative-pressure treatment device for skin wounds according to claim 1, characterized by further comprising a closing device for skin incision; the closing device for skin incision is connected to the base straps (3) at two sides of the skin wound.
